(19) United States
(12) Patent Application Publication
(10) Pub. No.: US 2015/0225380 A1
(43) Pub. Date: Aug. 13, 2015

(54) Novel Method to Obtain Olmesartan Medoxomil With Reduced Particle Size

(71) Applicant: LUPIN LIMITED, Pune, Maharashtra (IN)

(72) Inventors: Govind Dnyanoba Ausekar, Pune (IN); Radhakrishna Bhikaji Shivdavkar, Pune (IN); Himanshu Madhav Godbole, Pune (IN); Rajendra Vishwanath Firke, Pune (IN); Girij Pal Singh, Pune (IN)

(73) Assignee: LUPIN LIMITED, Pune, Maharashtra (IN)

(21) Appl. No.: 14/422,801

(22) PCT Filed: Jul. 26, 2013
(86) PCT No.: PCT/IB2013/056142

§ 371 Date: Feb. 20, 2015

(30) Foreign Application Priority Data
Aug. 22, 2012 (IN) 962/KOL/2012

(51) Int. Cl. C07D 405/14

(52) U.S. Cl. CPC C07D 405/14

(57) ABSTRACT

The present invention provides a novel method to obtain olmesartan medoxomil (I) with a particle size distribution of less than 30 μm comprising: dissolving olmesartan medoxomil (I) in a solvent; adding seed crystals of olmesartan medoxomil (I), followed by isolation.

### FIELD OF INVENTION

The present invention relates to a method to obtain olmesartan medoxomil having a particle size distribution of less than 30 μm.

### BACKGROUND OF THE INVENTION

Olmesartan medoxomil is chemically known as 4-(1-hydroxy-1-methylethyl)-2-propyl-1-[[2′-(1H-tetrazol-5-yl) [1, 1′-biphenyl]-4-yl] methyl]-1H-imidazole-5-carboxylic acid (5-methyl-2-oxo-1,3-dioxol-4-yl)methyl ester and represented by formula I

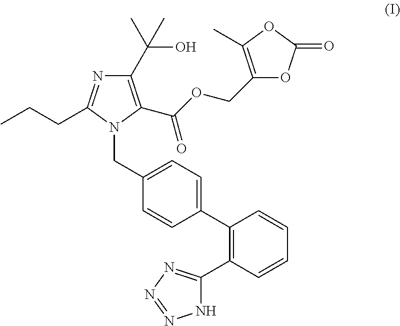 [SMILES: CCCc1nc(C(C)(C)O)c(C(=O)OCc2oc(=O)oc2C)n1Cc1ccc(-c2ccccc2-c2nnn[nH]2)cc1]

Olmesartan medoxomil (I) is a prodrug that is selective AT₁ subtype angiotensin II receptor antagonist and pharmaceutically used as an antihypertensive for the treatment and prophylaxis of hypertension. Formulation of Olmesartan medoxomil (I) are provided in tablet form. The particle size and specific surface area of the active ingredient used for drug preparation significantly affects medicinal effects and hence it is important to employ of olmesartan medoxomil (I) with suitable particle size for formulation.

When producing medicament containing olmesartan medoxomil (I) it is required that bioavailability of the drug should be in constant range from the standard value and since bioavailability is correlated with dissolution profile it is important to control dissolution profile. In general the dissolution properties can be improved by using drug substances of a pharmaceutical compound so as to have small particle diameter. Therefore, particle size of the drug needs to be controlled.

Olmesartan medoxomil (I) was first disclosed in U.S. Pat. No. 5,616,599, along with its process for preparation, however this patent does not provide any information about the particle size of the crystals obtained.

Sankyo Research Institute Annual Report, vol. 55, p. 1-91, 2003 provides physiochemical properties of olmesartan medoxomil (I) but does not provide any data for particle size distribution or specific surface area.

PCT application WO 2007/047838provides Olmesartan medoxomil (I) with particle size of D₁₀ less than about 50 μm, or less than about 30 μm; D₅₀ less than about 150 μm, or less than about 100 μm; and D₉₀ less than about 250 μm, or less than about 200 μm. The application however does not provide any specific method to obtain the mentioned particle size.

The application US 20060281800 A1 discloses polymorph Form G of olmesartan medoxomil (I) with D₅₀ and D₉₀ particle size of less than about 400 microns, preferably less than about 200 microns, more preferably less than about 150 microns, still more preferably less than about 50 microns and most preferably less than about 15 microns. The particle sizes can be obtained by, for example, by milling, grinding, micronizing or other particle size reduction method known in the art.

The U.S. Pat. No. 7,943,779 states that it is important to control size of particles of olmesartan medoxomil (I) during its preparation and if bigger particles, e.g. with an average diameter of above 100 μm are obtained they need to be milled or processed in any other way which reduces particle size, prior to their application in pharmaceutical formulations. The following parameters are defined to control particle size distribution: 10% of particles smaller than 20 μm, preferably smaller than 15 μm; 50% of particles smaller than 80 μm, preferably smaller than 50 μm, 90% of particles smaller than 170 μm, preferably smaller than 140 μm.

The application US 2010/0062070 A1 provides olmesartan medoxomil (I) having a particle diameter at 90% cumulative volume of 75 μm, or less and states that it can be produced by pulverizing crystals having a larger particle diameter. The methods to pulverize crystals could be knife type, hammer type, pin type, jet type and the like.

PCT application WO 2011/045760 provides pharmaceutical composition that includes micronized particles of olmesartan medoxomil (I) having d_{0.9} less than 50 μm and one or more pharmaceutically acceptable excipients.

The prior art methods suggest that smaller particle size of olmesartan medoxomil (I) is preferred for formulation and hence olmesartan medoxomil is micronized by using conventional methods like milling involving various techniques.

However, it is been observed by the present inventors that olmesartan medoxomil (I) has very low minimum ignition energy of less than 3 mJ and has very high powder resistivity making it unsafe for milling. If pulverization has to carried out extreme precaution need to be taken with respect to static charge dissipation and specially designed equipment would be required to avert explosion.

Therefore, a need arises to develop a safe industrial process for producing olmesartan medoxomil (I) with smaller particle size distribution without using any of the particle size reducing technique, which is of a high risk in this case.

The present invention provides method which gives olmesartan medoxomil (I) with particle size distribution of less than 30 μm.

### SUMMARY OF THE INVENTION

The present invention provides novel method to obtain olmesartan medoxomil (I) with particle size distribution of less than 30 μm making it suitable for formulation.

The present invention provides a novel method to obtain olmesartan medoxomil (I) with a particle size distribution of less than 30 μm comprising: dissolving olmesartan medoxomil (I) in a solvent; adding seed crystals of olmesartan medoxomil (I), followed by isolation.

### DETAILED DESCRIPTION OF THE INVENTION

In a preferred embodiment, the present invention provides a novel method to obtain olmesartan medoxomil (I) with a particle size distribution of less than 30 μm comprising:

a) dissolving olmesartan medoxomil (I) in a solvent,

b) adding seed crystals of olmesartan medoxomil (I), and

c) isolation.

The solvent is selected from ketones like acetone, methyl ethyl ketone, methyl isobutyl ketone, cyclohexanone, cycloheptanone etc.; esters such as ethyl acetate, butyl acetate etc.; chlorinated hydrocarbons such as dichloromethane, chloroform, ethylene dichloride etc.; nitriles such as acetonitrile, propionitrile etc.; ethers such as diethyl ether, diisopropyl ether, t-butyl methyl ether, tetrahydrofuran, dioxan etc.; hydrocarbons such as hexane, heptane, cyclohexane, cycloheptane, benzene, toluene, xylene etc.; or mixtures thereof; preferably acetone.

Olmesartan medoxomil (I) is dissolved in the solvent by heating, in the temperature range of 40-100° C., preferably at 40-60° C. Solution is cooled to 20-30° C.

The seed crystals are added to the solution at a temperature of 20-30° C. The seed crystals have a particle size of d₉₀ less than 20 μm, preferably less than 10 μm.

The agitation speed of the solution is not particularly limited but may be employed in the range of 100-500 rpm, preferably 200-300 rpm.

Olmesartan medoxomil (I) is isolated by techniques known in art like filtration, evaporation, concentration of solvent etc.

Olmesartan medoxomil (I) crystals obtained exhibit a particle size distribution of less than 30 μm. The following parameters are defined to control particle size distribution: d₁₀ less than 5 μm, preferably less than 2 μm; d₅₀ less than 15 μm, preferably less than 10 μm, d₉₀ less than 30 μm, preferably less than 20 μm.

Olmesartan medoxomil (I) utilized in the present invention can be produced in accordance with the method described in U.S. Pat. No. 5,616,599 and other documents.

Olmesartan medoxomil (I) of the present invention can be formulated into tablet by methods known in the art. Further, this tablet formulation can be prepared directly using olmesartan medoxomil without the necessity of reducing the particle size.

The manufacture of olmesartan medoxomil (I) as per the process of present invention has the following advantages over the prior art methods:

a. Does not utilize high risk micronization process therefore it is safer and suitable for plant scale manufacture,

b. Avoids use of specially designed equipment for micronization process,

c. Avoids yield loss due to micronization,

d. Time and energy efficient since it avoids micronization step.

The present invention is further illustrated by the following representative examples and does not limit the scope of the invention.

The particle size distribution of olmesartan medoxomil (I) is measured utilizing: Instrument model: Malvern; Dispersion unit: Hydro2000S (A); Particle refraction index of sample: 1.427; Absorption: 0.1; Dispersant refraction index: 1.468 and Size range: 0.020-2000 μm.

### EXAMPLE 1

### Preparation of Olmesartan Medoxomil (I)

A mixture of olmesartan medoxomil (12 g) and acetone (190 ml) was heated at about 55° C. to obtain a clear solution. The solution was cooled to about 25° C. and seed crystals of olmesartan medoxomil (0.12 g) were added. The mixture was stirred for 30 minutes and acetone (about 120 ml) was distilled out under vacuum. The slurry was cooled to 0-5° C. and stirred for 3 hours. The solid was filtered, washed with acetone and dried under vacuum. Yield 11 g (92%); particle size distribution: d₁₀ of 0.984 μm, d₅₀ of 8.484 μm, d₉₀ of 18.756 μm.

1. A method to obtain olmesartan medoxomil (I) with a particle size distribution of less than 30 μm comprising: 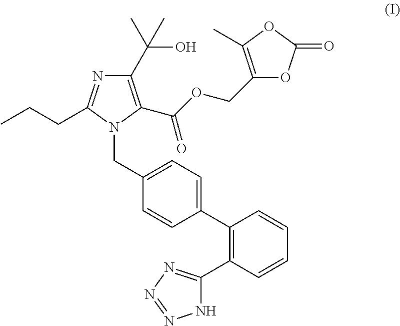 [SMILES: CCCc1nc(C(C)(C)O)c(C(=O)OCc2oc(=O)oc2C)n1Cc1ccc(-c2ccccc2-c2nnn[nH]2)cc1]
a) dissolving olmesartan medoxomil (I) in a solvent,
b) adding seed crystals of olmesartan medoxomil (I), and
c) isolation.

2. A method according to claim 1 wherein, solvent is selected from ketones such as acetone, methyl ethyl ketone, methyl isobutyl ketone, cyclohexanone, cycloheptanone; esters such as ethyl acetate, butyl acetate; chlorinated hydrocarbons such as dichloromethane, chloroform, ethylene dichloride; nitriles such as acetonitrile, propionitrile; ethers such as diethyl ether, diisopropyl ether, t-butyl methyl ether, tetrahydrofuran, dioxan; hydrocarbons such as hexane, heptane, cyclohexane, cycloheptane, benzene, toluene, xylene; or mixtures thereof.

3. A method according to claim 2 wherein, solvent is acetone.

4. A method according to claim 1 wherein, olmesartan medoxomil is dissolved by heating at a temperature of 40-100° C.

5. A method according to claim 4 wherein, olmesartan medoxomil is dissolved by heating at a temperature of 40-60° C.

6. A method according to claim 1 wherein, seed crystals of olmesartan medoxomil have a particles size of d₉₀ less than 20 μm.

7. A method according to claim 6 wherein, seed crystals of olmesartan medoxomil have a particles size of d₉₀ less than 10 μm.

8. A method according to claim 1 wherein, seed crystals of olmesartan medoxomil are added at a temperature of 20-30° C.

9. A method according to claim 1 wherein, olmesartan medoxomil is isolated by either filtration or evaporation or concentration of solvent.

10. A method according to claim 9 wherein, olmesartan medoxomil is isolated by filtration.

11. A method according to claim 1 wherein, olmesartan medoxomil (I) has particle size of d₁₀ less than 5 μm, d₅₀ less than 15 μm and d₉₀ less than 30 μm.

12. A method according to claim 11 wherein, olmesartan medoxomil (I) has particle size of d₁₀ less than 2 μm; d₅₀ less than 10 μm and d₉₀ less than 20 μm.
